(19) United States
(12) Patent Application Publication
(10) Pub. No.: US 2026/0133214 A1
(43) Pub. Date: May 14, 2026

(54) AUTOMATION DEVICE FOR IDENTIFYING AND COLLECTING BIOLOGICAL AGENTS

(71) Applicant: AGENCY FOR DEFENSE DEVELOPMENT, Daejeon (KR)

(72) Inventors: Kibong CHOI, Daejeon (KR); Jungho LEE, Daejeon (KR); Hyunsoo SEO, Daejeon (KR)

(21) Appl. No.: 19/301,567

(22) Filed: Aug. 15, 2025

(30) Foreign Application Priority Data
Nov. 11, 2024 (KR) 10-2024-0159310

(51) Int. Cl. G01N 35/04; G01N 35/00; G01N 35/10

(52) U.S. Cl. CPC G01N 35/04; G01N 35/00732; G01N 35/1011; G01N 2035/00475; G01N 2035/00801; G01N 2035/00831; G01N 2035/0415; G01N 2035/1027

(57) ABSTRACT

Provided herein is an automation device for identifying and collecting biological agents, which improves user convenience and enhances operational efficiency by integrating the collection and identification processes. The automation device includes: a collection unit including a movable filter tray; and an identification unit disposed apart from the collection unit and configured to wash and store collected biological agents, where a filter assembly may be mounted on the filter tray, the filter assembly being configured to collect biological agents by passing external air through a filter, and the filter coupled inside the filter assembly may be configured to automatically rotate as the filter tray moves to the identification.

### CROSS-REFERENCE TO RELATED APPLICATION

This application claims priority to Korean Patent Application No. 10-2024-0159310 filed on November 11, 2024, and all the benefits accruing therefrom under 35 U.S.C. §119, the contents of which are incorporated herein by reference in their entirety.

### BACKGROUND OF THE DISCLOSURE

### Field of the Disclosure

The present disclosure relates to an automation device for identifying and collecting biological agents. In particular, the present disclosure relates to a compact biological particle identification device capable of miniaturization by enabling a single device to: collect biological particles through a simple structure; aspirate a cleaning solution to separate the biological particles from a filter and discharge a sample solution; inject the discharged sample solution into a plurality of ticket modules; and identify biological particles through image analysis of the ticket modules.

### DESCRIPTION OF THE RELATED ART

Biological weapons used for terrorism and military purposes are highly efficient to produce and can be dispersed from tens of kilometers away, causing infection in humans even at low exposure levels. These agents replicate within the body, undergo an incubation period, and eventually cause disease, potentially leading to widespread transmission that threatens the lives of an unspecified large population. Therefore, in the event of conflict with hostile forces or a terrorist attack involving the deployment or suspected deployment of biological weapons, rapid detection of such weapons must take precedence. In addition, considering the wide-area contamination caused by wind direction and speed in the environment where dispersion occurs, identification devices for biological particles are being developed with a focus on miniaturization and lightweight design to facilitate transport and mobility.

For example, the compact biological particle identification device disclosed in Korean Registered Patent Publication No. 10-2212748 is configured to generate a sample containing biological particles by mounting a filter module, which has collected biological particles from surveillance equipment or collectors, and separating the biological particles from the filter module through spraying of a cleaning solution. An antigen-antibody reaction is then performed on the sample, followed by image analysis, to enable the detection of the biological particles.

These devices, in order to operate effectively even in low-concentration biological particle contamination environments that are widely dispersed, typically collect a sufficient amount of sample on a sampling filter via a separate collection device before transferring the sample to the identification device to prepare a sample solution. However, this process requires the transfer of the filter between the collector and the identifier, which can result in biological particles being scattered into the surroundings and exposed to the external environment, leading to contamination issues.

### Related Patent Document

Korean Registered Patent Publication No. 10-1310116

Korean Registered Patent Publication No. 10-2212748

### SUMMARY OF THE DISCLOSURE

The purpose of the present disclosure, which aims to solve the aforementioned conventional problems, is to provide an automation device for identifying and collecting biological agents, which improves user convenience and enhances operational efficiency by integrating the collection and identification processes. This is achieved by enabling a continuous and automated process is carried out sequentially within a single device having a simple structure. That is, in an automated and sequential process, biological particles are collected, a sample solution is prepared, and the identification process is performed by injecting the sample into a ticket module and a transferred sample container.

In order to achieve the purpose, an aspect of the present disclosure provides an automation device for identifying and collecting biological agents, comprising: a collection unit including a movable filter tray; and an identification unit disposed apart from the collection unit and configured to wash and store collected biological agents,

a filter assembly may be mounted on the filter tray, the filter assembly being configured to collect biological agents by passing external air through a filter, and

the filter coupled inside the filter assembly may be configured to automatically rotate as the filter tray moves to the identification unit for identification of the collected biological agents.

In some exemplary embodiments, the filter tray may be configured to move along two axes, including a left-right direction and an up-down direction.

In some exemplary embodiments, the identification unit may includes a ticket tray on which a collection container is mounted, and

the ticket tray may be configured to move along three axes, including a left-right direction, a front-rear direction, and an up-down direction.

In some exemplary embodiments, the filter assembly may comprise:

a filter housing configured to hold the filter; and

a filter fixing bracket to which the filter housing is rotatably coupled,

the filter housing may be configured to rotate via a rotating shaft connected to a driving shaft.

In some exemplary embodiments, a collection port may be detachably connected to an upper side of the filter assembly, the collection port being configured to collect external air,

a dummy filter may be mounted on the filter tray, the dummy filter being spaced apart from the filter assembly, and

after the filter tray moves to the identification unit, the dummy filter may be coupled to the collection port.

In some exemplary embodiments, the identification unit may include a movable ticket tray and a collection container mounted on the ticket tray, and

when the filter tray moves to the identification unit, the filter assembly and the collection container may be brought into alignment and coupled to each other.

In some exemplary embodiments, after the filter coupled inside the filter assembly automatically rotates, a cleaning solution may be dispensed into the filter assembly to wash the biological agents into the collection container.

In some exemplary embodiments, the identification unit may include a movable ticket tray, a collection container mounted on the ticket tray, and a needle assembly, and

the ticket tray may be configured to move such that the filter assembly and the needle assembly are brought into alignment and coupled to each other.

In some exemplary embodiments, after the filter assembly and the needle assembly are coupled to each other, the ticket tray may be configured to move such that the filter assembly is brought into alignment with the collection container, and

the needle assembly may be configured to aspirate a solution containing biological agents from the collection container.

In some exemplary embodiments, the needle assembly may comprise: a connector configured to be coupled to the filter assembly; and a needle configured to aspirate and inject a solution,

and the needle may be detachably attached to the connector.

In some exemplary embodiments, the identification unit may include a movable ticket tray, and

an identification ticket may be mounted on the ticket tray, the identification ticket being configured to perform an antigen-antibody reaction for the biological agents.

In some exemplary embodiments, the automation device may further comprise: a camera assembly configured to capture an image of the identification ticket; and an image processing module configured to analyze the captured image.

In some exemplary embodiments, the ticket tray may be configured to move such that the camera assembly is aligned to capture an image of the identification ticket, and

the image processing module may be configured to analyze the image captured by the camera assembly to identify a type and quantity of the biological agents.

In some exemplary embodiments, the identification unit may include a movable ticket tray, and

a transferred sample container may be mounted on the ticket tray, the transferred sample container being configured to store and transfer a sample of the biological agents.

In some exemplary embodiments, the ticket tray may be configured to move such that the filter assembly is aligned with the transferred sample container, and

the filter assembly may be configured to inject the biological agents into the transferred sample container.

In addition, in order to achieve the purpose, another aspect of the present disclosure provides an automation device for identifying and collecting biological agents, comprising: a collection port configured to collect external air; a filter assembly configured to collect biological agents by passing the external air collected by the collection port through a filter; a filter tray on which the filter assembly is mounted, the filter tray being configured to be movable; and a ticket tray disposed apart from the filter tray, the ticket tray being movable and having a collection container mounted thereon, the collection container being configured to store the biological agents,

the filter coupled inside the filter assembly may be configured to automatically rotate as the filter tray moves to the ticket tray for identification of the collected biological agents.

In addition, in order to achieve the purpose, still another aspect of the present disclosure provides an automation device for identifying and collecting biological agents, comprising: a collection port configured to collect external air; a filter assembly detachably connected to the collection port and configured to collect biological agents by passing the external air collected by the collection port through a filter; a filter tray on which the filter assembly and a dummy filter are mounted, the filter tray being configured to be movable; and a ticket tray disposed apart from the filter tray, the ticket tray being movable and having a collection container mounted thereon, the collection container being configured to store the biological agents,

as the filter tray moves to the ticket tray for identification of the biological agents, the filter assembly may be disconnected from the collection port, and

after the filter tray moves to the ticket tray, the dummy filter may be connected to the collection port.

Specific details of other exemplary embodiments are included in "Details for carrying out the invention" and accompanying "drawings".

Advantages and/or features of the present disclosure, and a method for achieving the advantages and/or features will become obvious with reference to various exemplary embodiments to be described below in detail together with the accompanying drawings.

However, the present disclosure is not limited only to a configuration of each exemplary embodiment disclosed below, but may also be implemented in various different forms. The respective exemplary embodiments disclosed in this specification are provided only to complete disclosure of the present disclosure and to fully provide those skilled in the art to which the present disclosure pertains with the category of the present disclosure, and the present disclosure will be defined only by the scope of each claim of the claims.

According to the configuration, combination, and use relationships described in the present specification, the present disclosure provides the following effects.

The present disclosure enables automation of the identification process, including collecting biological particles, preparing a sample solution, and injecting the sample into a ticket module and a transferred sample container, as a continuous process within a single device having a simple structure. Accordingly, the present disclosure eliminates the need to transfer the filter between a collector and an identifier, thereby improving user convenience and integrating the collection and identification processes to enhance operational efficiency.

In addition, the present disclosure allows the filter module such as a surveillance device or the like to be transported in a sealed state via the filter assembly, enabling a continuous collection-identification process while preventing contamination of the filter and surrounding environment, and facilitating easy transport and installation of the filter module.

Furthermore, by improving the structure of the filter assembly, the present disclosure enables the use of commercially available disposable needles in the coupled needle assembly, making replacement easier and providing an economic advantage by reducing the consumable costs associated with conventional one-piece filter assemblies.

### BRIEF DESCRIPTION OF THE DRAWINGS

FIG. 1 is a perspective view illustrating an automation device for identifying and collecting biological agents according to an exemplary embodiment of the present disclosure.

FIG. 2 is a perspective view illustrating the moving direction of a filter tray in the automation device for identifying and collecting biological agents according to an exemplary embodiment of the present disclosure.

FIG. 3 is a perspective view illustrating the moving direction of a ticket tray in the automation device for identifying and collecting biological agents according to an exemplary embodiment of the present disclosure.

FIG. 4 is a view illustrating the configuration of a filter assembly mounted on the filter tray in the automation device for identifying and collecting biological agents according to an exemplary embodiment of the present disclosure.

FIG. 5 is a view illustrating the configuration of a filter housing in the automation device for identifying and collecting biological agents according to an exemplary embodiment of the present disclosure.

FIG. 6 is a view illustrating the rotation configuration of the filter assembly in the automation device for identifying and collecting biological agents according to an exemplary embodiment of the present disclosure.

FIG. 7 is a view illustrating the configuration of a needle assembly in the automation device for identifying and collecting biological agents according to an exemplary embodiment of the present disclosure.

FIGS. 8 to 18 are views illustrating the operation procedure of the automation device for identifying and collecting biological agents according to an exemplary embodiment of the present disclosure.

### DETAILED DESCRIPTION OF THE DISCLOSURE

Before describing the present disclosure in detail, the terms or words used in this specification should not be construed as being unconditionally limited to their ordinary or dictionary meanings, and in order for the inventor of the present disclosure to describe his/her disclosure in the best way, concepts of various terms may be appropriately defined and used, and furthermore, the terms or words should be construed as means and concepts which are consistent with a technical idea of the present disclosure.

That is, the terms used in this specification are only used to describe preferred embodiments of the present disclosure, and are not used for the purpose of specifically limiting the contents of the present disclosure, and it should be noted that the terms are defined by considering various possibilities of the present disclosure.

Further, in this specification, it should be understood that, unless the context clearly indicates otherwise, the expression in the singular may include a plurality of expressions, and similarly, even if it is expressed in plural, it should be understood that the meaning of the singular may be included.

In the case where it is stated throughout this specification that a component "includes" another component, it does not exclude any other component, but may further include any other component unless otherwise indicated.

Furthermore, it should be noted that when it is described that a component "exists in or is connected to" another component, this component may be directly connected or installed in contact with another component, and in inspect to a case where both components are installed spaced apart from each other by a predetermined distance, a third component or means for fixing or connecting the corresponding component to the other component may exist, and the description of the third component or means may be omitted.

On the contrary, when it is described that a component is "directly connected to" or "directly accesses" to another component, it should be understood that the third element or means does not exist.

Similarly, it should be construed that other expressions describing the relationship of the components, that is, expressions such as “between” and “directly between” or “adjacent to” and “directly adjacent to” also have the same purpose.

In addition, it should be noted that if terms such as "one side surface", "other side surface", "one side", "other side", "first", "second", etc., are used in this specification, the terms are used to clearly distinguish one component from the other component and a meaning of the corresponding component is not limited used by the terms.

Further, in this specification, if terms related to locations such as "upper", "lower", "left", "right", etc., are used, it should be understood that the terms indicate a relative location in the drawing with respect to the corresponding component and unless an absolute location is specified for their locations, these location-related terms should not be construed as referring to the absolute location.

Further, in this specification, in specifying the reference numerals for each component of each drawing, the same component has the same reference number even if the component is indicated in different drawings, that is, the same reference number indicates the same component throughout the specification.

In the drawings attached to this specification, a size, a location, a coupling relationship, etc. of each component constituting the present disclosure may be described while being partially exaggerated, reduced, or omitted for sufficiently clearly delivering the spirit of the present disclosure, and thus the proportion or scale may not be exact.

Further, hereinafter, in describing the present disclosure, a detailed description of a configuration determined that may unnecessarily obscure the subject matter of the present disclosure, for example, a detailed description of a known technology including the prior art may be omitted.

Moreover , one or more “unit” and/or “module” described in this specification can be implemented via a non-transitory memory (not shown) and a processor (not shown). The memory is configured to store data concerning algorithms designed to control the operation of system components according to exemplary embodiments of the present disclosure, or software instructions that implement these algorithms. The processor is configured to perform the operations described below using the data stored in the memory. Here, the memory and the processor may be implemented as separate chips. Alternatively, the memory and the processor may be implemented as a single integrated chip. The processor may take the form of one or more processors.

Furthermore, in the specification of the present disclosure, terms such as “unit,” “device,” “module,” “means,” and “apparatus,” if used, refer to a unit capable of processing one or more functions or operations and should be understood to be implementable in hardware, software, or a combination of hardware and software.

As will be understood by those skilled in the art, the realization of all or some of the steps of the above exemplary embodiments may be accomplished through hardware, or may be accomplished by directing the relevant hardware through a computer program. The computer program may include instructions for executing some or all of the steps of the method, the computer program may be stored on a readable storage medium, and the storage medium may be any form of storage medium.

Hereinafter, exemplary embodiments of the present disclosure will be described in detail with reference to related drawings.

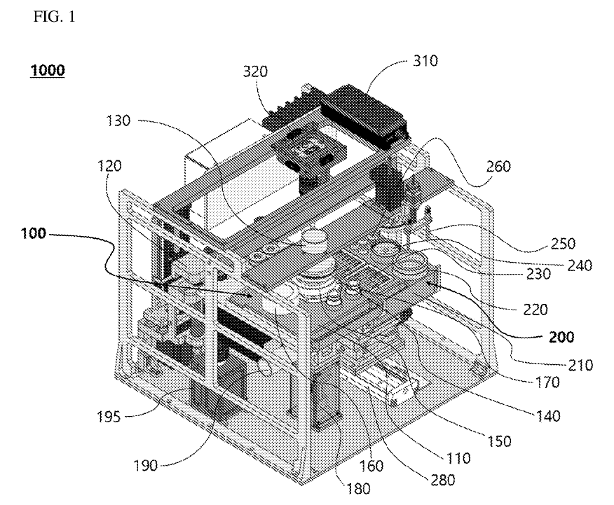

FIG. 1 is a perspective view illustrating an automation device 1000 for identifying and collecting biological agents according to an exemplary embodiment of the present disclosure.

As illustrated in FIG. 1, an automation device for identifying and collecting biological agents 1000 according to an exemplary embodiment of the present disclosure may include a collection unit 100 comprising a movable filter tray 110, and an identification unit 200 disposed apart from the collection unit 100 and configured to wash and store the collected biological agents.

A filter assembly 150, which is configured to collect biological agents by passing external air through a filter 156, may be mounted on the filter tray 110.

As the filter tray 110 moves toward the identification unit 200 for identification of the biological agents, the filter 156 coupled inside the filter assembly 150 may be automatically rotated.

Through this automatic rotation of the filter 156 coupled inside the filter assembly 150, the biological agents collected on the upper side of the filter 156 are transferred to the lower side. Subsequently, when a cleaning solution is dispensed, the biological agents are washed down into the collection container 220, eliminating the need for the filter 156 to be physically transferred between the collector and the identifier.

Accordingly, the device improves user convenience and integrates the collection and identification processes, thereby enhancing operational efficiency.

Moreover, since the filter 156 is transported in a sealed state, contamination of the filter 156 and the surrounding environment is prevented, and the filter module can be easily transported and mounted.

The collection unit 100 may include a filter tray 110, a horizontal driving unit 120, a rotational driving unit 125, a collection port 130, a filter holder 140, a filter assembly 150, a dummy filter 160, a cleaning solution container 170, a vertical driving unit 180, a discharge port 190, a rotary valve 195, and the like.

The identification unit 200 may include a ticket tray 210, a collection container 220, an identification ticket 230, a needle assembly 240, a cleaning solution inlet 250, a transferred sample container 260, a ticket tray path 280, and the like.

In addition, the automation device 1000 for identifying and collecting biological agents according to an exemplary embodiment of the present disclosure may further include an image processing module 310 for capturing images and a camera assembly 320 for capturing images of biological agents.

The structures and operating methods of the components of the automation device 1000 for identifying and collecting biological agents as described above will be described in detail below with reference to the accompanying drawings.

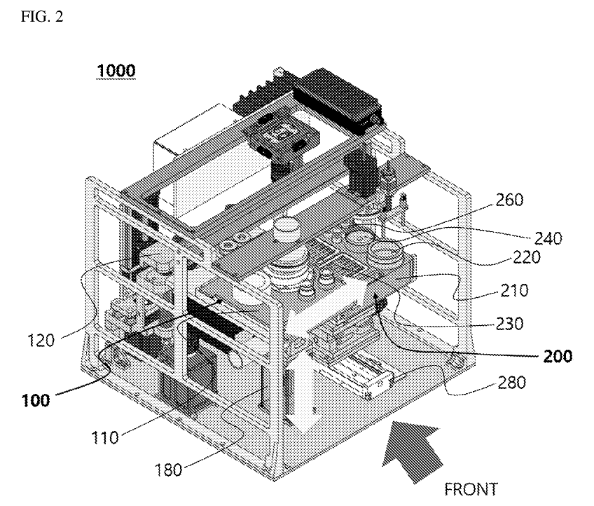

FIG. 2 is a perspective view illustrating the moving direction of a filter tray 110 in the automation device 1000 for identifying and collecting biological agents according to an exemplary embodiment of the present disclosure.

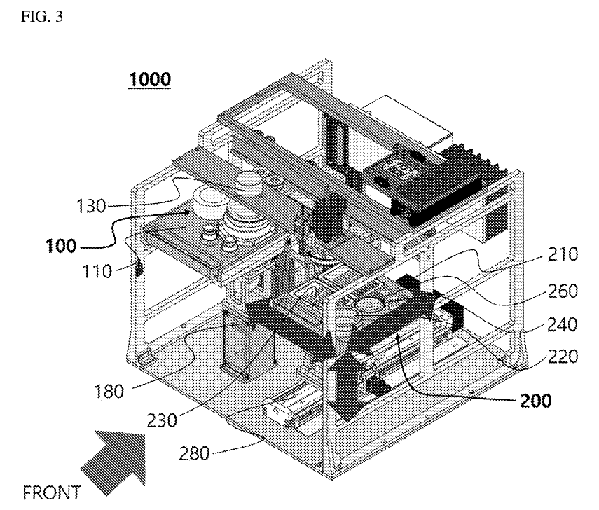

In addition, FIG. 3 is a perspective view illustrating the moving direction of a ticket tray 210 in the automation device 1000 for identifying and collecting biological agents according to an exemplary embodiment of the present disclosure.

As illustrated in FIGS. 2 and 3, in the automation device 1000 for identifying and collecting biological agents according to an exemplary embodiment of the present disclosure, the filter tray 110 may be capable of two-axis movement including the left-right direction and the up-down direction.

In addition, the identification unit 200 may include a ticket tray 210 on which a collection container 220 is mounted, and the ticket tray 210 may be capable of three-axis movement including the left-right direction, the front-rear direction, and the up-down direction.

The collection unit 100 may include a horizontal drive unit 120 and a vertical drive unit 180 for enabling the two-axis movement of the filter tray 110.

Each of the horizontal drive unit 120 and the vertical drive unit 180 may be driven by a motor to move the filter tray 110 along the two axes in the left-right and up-down directions when viewed from the front.

Similarly, the identification unit 200 may also include a horizontal drive unit 120 and a vertical drive unit 180 for moving the ticket tray 210 in the left-right and up-down directions when viewed from the front.

Furthermore, the identification unit 200 may include a ticket tray transfer path 280 for moving the ticket tray 210 in the front-rear direction when viewed from the front, thereby enabling three-axis movement of the ticket tray 210.

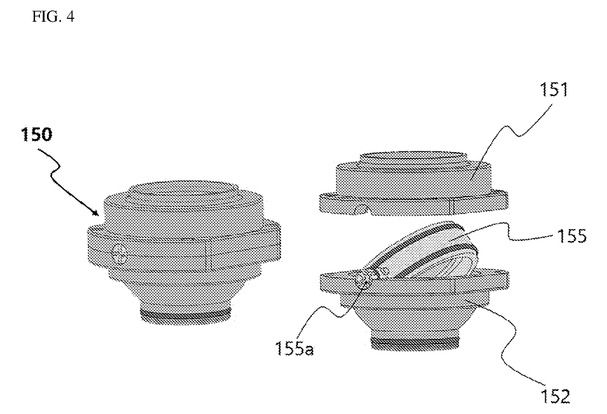

FIG. 4 is a view illustrating the configuration of a filter assembly 150 mounted on the filter tray 110 in the automation device 1000 for identifying and collecting biological agents according to an exemplary embodiment of the present disclosure.

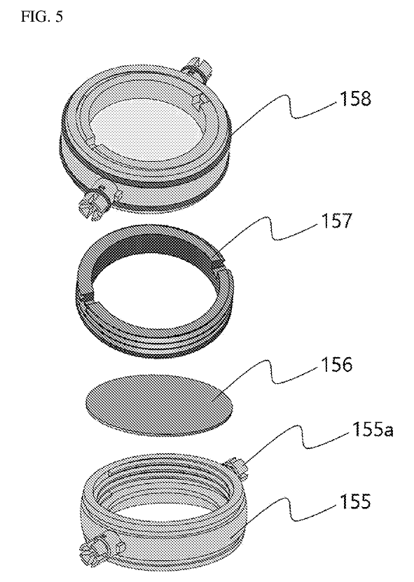

In addition, FIG. 5 is a view illustrating the configuration of a filter housing 155 in the automation device 1000 for identifying and collecting biological agents according to an exemplary embodiment of the present disclosure.

Referring to FIGS. 4 and 5, in an exemplary embodiment of the present disclosure, the filter assembly 150 may include a filter housing 155 configured to secure a filter and a filter fixing bracket 157 to which the filter housing 155 is rotatably coupled.

The filter housing 155 may be configured to rotate via a rotation shaft 155a connected to a drive shaft.

That is, the filter housing 155 may have the rotation shaft 155a formed thereon, and the rotation shaft 155a may be connected to the drive shaft so that the filter housing 155 can rotate.

In an exemplary embodiment of the present disclosure, the drive shaft may be a rotation driving unit 125 provided in the collection unit 100.

The rotation driving unit 125 may be driven by a motor and configured to rotate the filter housing 155 by being connected to the rotation shaft 155a of the housing.

More specifically, the filter assembly 150 may include an upper case 151, a lower case 152, the filter housing 155, a filter 156, and the filter fixing bracket 157.

To ensure the airtightness of the filter assembly 150, a sealing ring 158 may be disposed on the outer circumferential surface of the filter housing 155 to maintain a sealed state.

The filter fixing bracket 157 may be installed on the inner circumferential surface of the filter housing 155 to stably secure the filter 156, and the filter 156 for collecting biological agents may be coupled to the filter fixing bracket 157.

In addition, rotation shafts 155a may be protrudingly formed at both ends of the outer circumferential surface of the filter housing 155 to enable the rotation of the filter housing 155.

The filter housing 155 may be installed between the upper case 151 and the lower case 152 of the filter assembly 150 so as to be rotatable about the rotation shaft 155a, and the interior of the filter assembly 150 may be sealed by coupling the upper case 151 and the lower case 152.

The filter assembly 150 thus assembled may be mounted on the filter tray 110.

When the filter tray 110, on which the filter assembly 150 is mounted, is installed in the collection unit 100, the upper case 151 of the filter assembly 150 may be connected to a filter holder 140, which is coupled to the collection port 130 of the collection unit 100, and the lower case 152 may be connected to the discharge port 190 of the collection unit 100.

When the filter assembly 150 is connected to the collection port 130 and the discharge port 190 in this manner, the collection unit 100 may inhale external air through the collection port 130 at the upper side, biological agents contained in the external air may be collected on the upper side of the filter 156, and air that has passed through the filter 156 may be discharged to the outside through the discharge port 190.

In addition, in an exemplary embodiment of the present disclosure, the collection unit 100 may include a rotary valve 195.

The rotary valve 195 serves to control or block the flow of air. After external air is drawn in through the collection port 130, biological agents are collected by the filter 156, and the purified air is discharged through the discharge port 190. In this process, the rotary valve 195 regulates the flow of air, thereby optimizing the particle collection efficiency of the filter 156.

Moreover, when the filter 156 needs to be replaced or maintained, the rotary valve 195 may block the discharge port 190 to stop the external air flow and maintain constant pressure within the collection unit 100. By adjusting the timing of collection and discharge in the automation device 1000 for identifying and collecting biological agents according to the present disclosure, the rotary valve 195 can contribute to improving the operational efficiency and safety of the device.

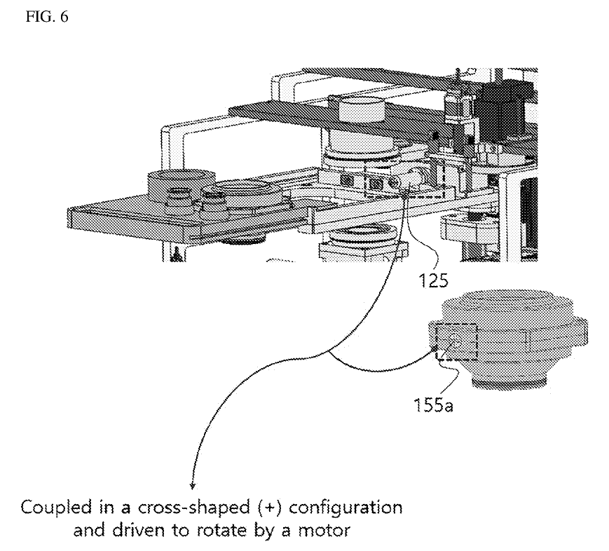

FIG. 6 is a view illustrating the rotation configuration of the filter assembly 150 in the automation device 1000 for identifying and collecting biological agents according to an exemplary embodiment of the present disclosure.

Referring to FIG. 6, the rotation driving unit 125 provided in the collection unit 100 may be formed with a protrusion or groove in a "+" shape, and the rotation shaft 155a formed on the filter housing 155 may be provided with a groove or protrusion in a "+" shape, respectively.

The filter assembly 150 is mounted on the filter tray 110, and when the filter tray 110 is mounted on the collection unit 100, the rotation shaft 155a formed on the filter housing 155 of the filter assembly 150 may be engaged with the rotation driving unit 125 of the collection unit 100.

In this configuration, when the filter tray 110 is horizontally moved to the identification unit 200 for the identification of biological agents, the rotation driving unit 125 may also be rotated by a motor to rotate the filter housing 155, which is coupled inside the filter assembly 150.

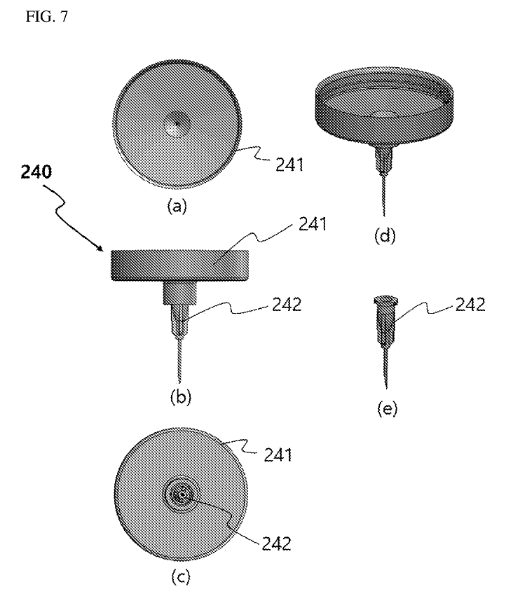

Furthermore, FIG. 7 is a view illustrating the configuration of a needle assembly 240 in the automation device 1000 for identifying and collecting biological agents according to an exemplary embodiment of the present disclosure.

Referring to FIGS. 1 to 7, in an exemplary embodiment of the present disclosure, the upper side of the filter assembly 150 is detachably connected to the collection port 130 for collecting external air, and a dummy filter 160 is mounted on the filter tray 110 spaced apart from the filter assembly 150. After the filter tray 110 moves to the identification unit 200, the dummy filter 160 may be connected to the collection port 130.

The dummy filter 160 may serve to prevent contamination by blocking external air from flowing into the automation device 1000 for identifying and collecting biological agents through the collection port 130 after the filter assembly 150 has moved to the identification unit 200.

In addition, the dummy filter 160 may be used for an airflow test or initialization process before the device transitions back to the collection mode. While the filter tray 110 moves to the identification unit 200 and prepares for a new operation cycle, it is possible to check whether the airflow within the device is maintained correctly through the dummy filter 160. This allows for the detection of any abnormalities in advance before entering the actual collection mode.

In an exemplary embodiment of the present disclosure, the identification unit 200 may include a movable ticket tray 210 and a collection container 220 mounted on the ticket tray 210. After the filter tray 110 moves to the identification unit 200 for the identification of biological agents, the filter assembly 150 and the collection container 220 may be aligned and coupled with each other.

At this time, the filter housing 155 coupled inside the filter assembly 150 may rotate 180 degrees by the rotation of the rotary driving unit 125, causing the biological agents, which have entered through the collection port 130 and collected on the upper side of the filter 156, to move to the lower side of the filter 156.

After the filter 156 coupled inside the filter assembly 150 is automatically rotated as described above, a washing solution may be introduced into the filter assembly 150 to wash the biological agents adhered to the lower side of the filter 156 into the collection container 220.

In an exemplary embodiment of the present disclosure, a washing solution container 170 may be mounted on the filter tray 110 at a position spaced apart from the filter assembly 150, and the washing solution container 170 may accommodate a washing solution.

The identification unit 200 may be provided with a washing solution inlet 250, and when the filter tray 110 moves to the identification unit 200 such that the filter assembly 150 and the collection container 220 are aligned with each other, the washing solution container 170 and the washing solution inlet 250 may also be configured to be aligned with each other.

After the washing solution container 170 and the washing solution inlet 250 are aligned, the washing solution inlet 250 may draw the washing solution stored in the washing solution container 170 and discharge the drawn washing solution into the filter assembly 150 through a nozzle, thereby washing the biological agents adhered to the lower side of the filter 156 into the collection container 220.

Through this process, the collection container 220 may accommodate a solution in which the washing solution and biological agents are mixed.

In an exemplary embodiment of the present disclosure, a needle assembly 240 may be mounted on the ticket tray 210 at a position spaced apart from the collection container 220.

After the solution in which the washing solution and biological agents are mixed is accommodated in the collection container 220, the ticket tray 210 may be moved such that the filter assembly 150 and the needle assembly 240 are aligned and coupled with each other.

Once the filter assembly 150 and the needle assembly 240 are coupled, the ticket tray 210 may move again to re-align the filter assembly 150 with the collection container 220, allowing the solution containing the washing solution and biological agents to be aspirated from the collection container 220 through the needle assembly 240 coupled to the filter assembly 150.

The needle assembly 240 is configured to aspirate the solution containing the washing solution and biological agents from the collection container 220 and perform the function of transferring and discharging it to the next stage.

That is, as illustrated in FIG. 7, the needle assembly 240 includes a connector 241 that can be coupled with the filter assembly 150 and a needle 242 configured to aspirate and inject a solution. The needle 242 may be detachably attached to the connector 241.

In an exemplary embodiment of the present disclosure, a commercially available disposable needle may be used as the needle 242 of the needle assembly 240.

In conventional biological particle identification devices, the filter assembly is configured as an integrated unit, requiring the entire filter assembly to be replaced in the event of contamination or damage.

In contrast, the automation device 1000 for identifying and collecting biological agents according to an exemplary embodiment of the present disclosure allows for easy attachment and detachment between the filter assembly 150 and the needle assembly 240, thereby reducing the consumable cost associated with using a conventional integrated filter assembly.

In addition, the needle 242 of the needle assembly 240 can be hygienically and conveniently replaced with a disposable needle, providing a sanitary advantage especially in biological particle identification devices that handle high-risk biological samples.

Furthermore, the automated attachment/detachment structure improves the operational efficiency of the device and minimizes manual intervention by operators, thereby enabling safe and fully automated continuous operation.

In an exemplary embodiment of the present disclosure, the ticket tray 210 of the identification unit 200 may include an identification ticket 230, which performs an antigen-antibody reaction for biological particles, and a transport sample container 260, which stores and transports a sample of biological particles. These components may be mounted at positions spaced apart from each other.

In addition, the automation device 1000 for identifying and collecting biological agents according to an exemplary embodiment of the present disclosure may further include a camera assembly 320 for capturing images of the identification ticket 230 and an image processing module 310 for analyzing the captured images.

The identification ticket 230 may include a plurality of ticket modules, each of which may contain an antibody for detecting biological particles.

When a sample containing biological particles reaches a ticket module, an antigen-antibody reaction occurs as the antibody binds to a specific antigen, enabling identification of the presence of the biological particles.

By providing a plurality of ticket modules within the device to detect biological particles through various antigen-antibody reactions as described above, the device enables accurate and rapid detection of biological particles, including a wide range of biological agents.

The transport sample container 260 stores biological particle samples and enables their transfer for additional analysis or delivery to external laboratories, if necessary. This ensures that even if preliminary detection of biological particles is performed within the device, further analysis for accurate verification is possible, while maintaining the storage condition of the biological particles to ensure the reliability of the analysis.

The camera assembly 320 is configured to capture images of each ticket module, and through the movement of the ticket tray 210, the identification ticket 230 including a plurality of ticket modules may be positioned below the camera assembly 320 so that each ticket module can be imaged and transmitted to the image processing module 310.

In addition, the camera assembly 320 may include a lighting module to ensure accurate image capture.

The image processing module 310 is configured to analyze the images of each ticket module to identify biological particles. In each ticket module, a reaction such as color development occurs due to an antigen-antibody reaction upon the introduction of a sample containing biological particles. The image processing module 310 analyzes the reaction and identifies the type and quantity of biological particles.

In such configuration as above, in an exemplary embodiment of the present disclosure, after the filter assembly 150 draws in the solution containing the washing liquid and biological particles from the collection container 220 via the needle assembly 240, the ticket tray 210 moves such that the filter assembly 150 aligns with the identification ticket 230, and the filter assembly 150 injects the solution into the identification ticket 230 through the needle assembly 240.

That is, after the filter assembly 150 automatically draws in the solution containing the washing liquid and biological particles from the collection container 220 via the needle assembly 240, and the ticket tray 210 automatically moves to align the filter assembly 150 with the identification ticket 230, the filter assembly 150 can inject the drawn solution into the identification ticket 230 via the needle assembly 240. In this step, biological particles can be identified through an antigen-antibody reaction.

Subsequently, the ticket tray 210 automatically moves again to align the filter assembly 150 with the transport sample container 260, and the filter assembly 150 injects the remaining solution into the transport sample container 260 via the needle assembly 240. Through this process, the sample can be stored in a state suitable for further analysis.

By storing the biological particle sample in the transport sample container 260, even when the biological particle is preliminarily detected within the device, additional analysis or external laboratory transfer for accurate confirmation can be performed. This enables the storage condition to be maintained and ensures the reliability of the analysis.

Furthermore, the ticket tray 210 automatically moves again to align the camera assembly 320 for image capture of the identification ticket 230, and the camera assembly 320 captures the image of the identification ticket 230. The image processing module 310 then analyzes the captured image to identify the type and quantity of the biological particles.

The camera assembly 320 captures the images of each ticket module included in the identification ticket 230, which comprises a plurality of ticket modules, and transmits them to the image processing module 310. The camera assembly 320 may include a lighting module to ensure accurate image capture.

The image processing module 310 is configured to analyze the captured images of each ticket module and identify the type and quantity of biological particles based on reactions such as color development resulting from the antigen-antibody reaction.

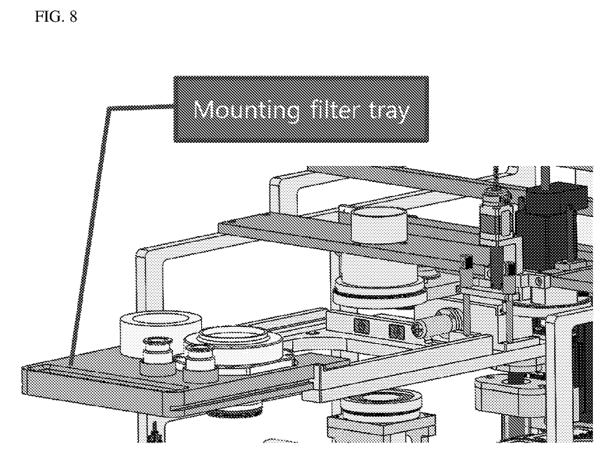

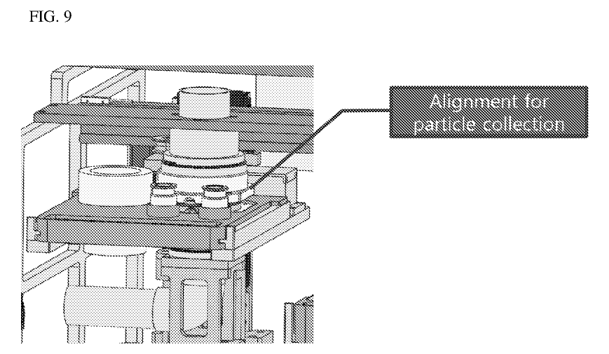

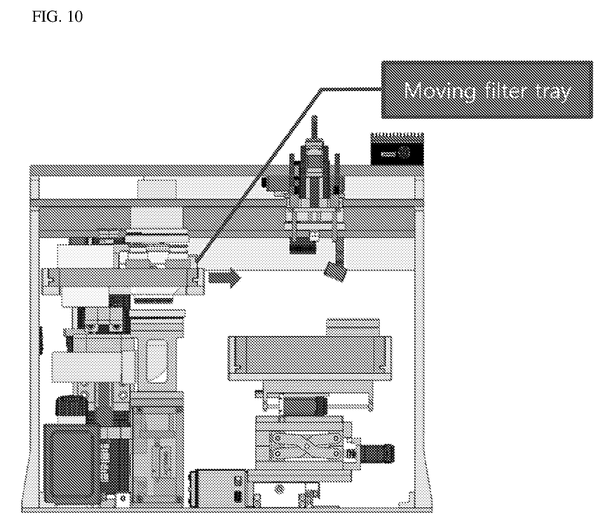

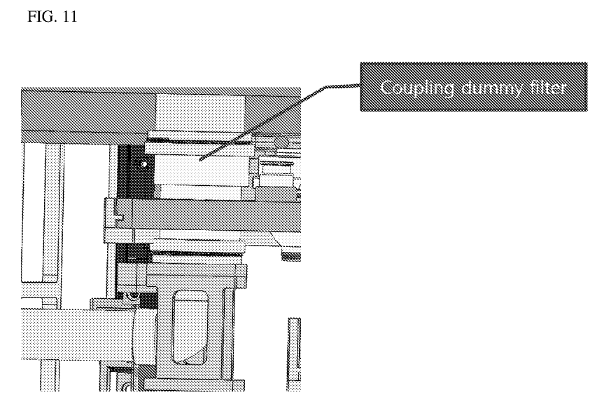

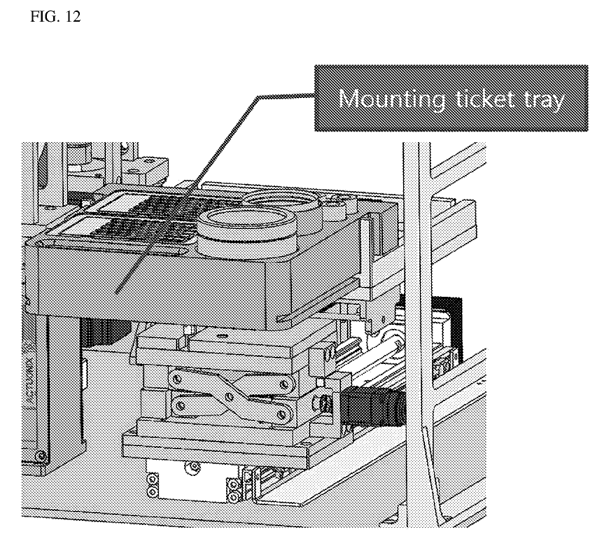

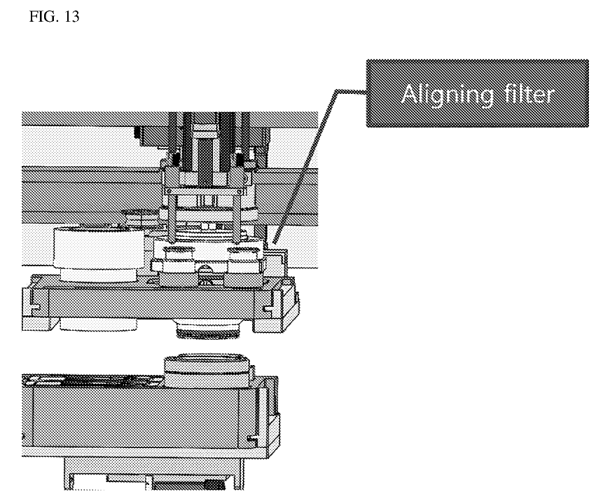

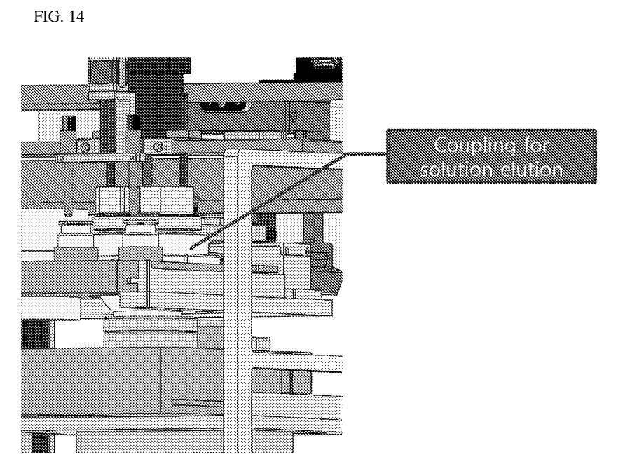

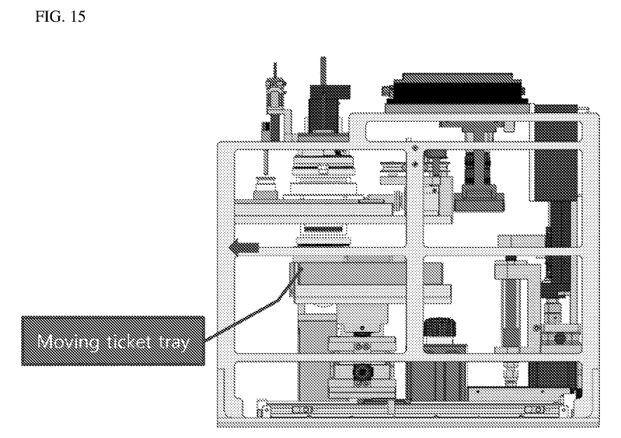

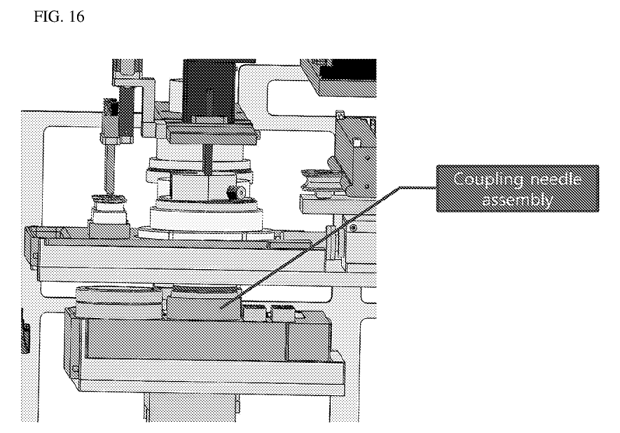

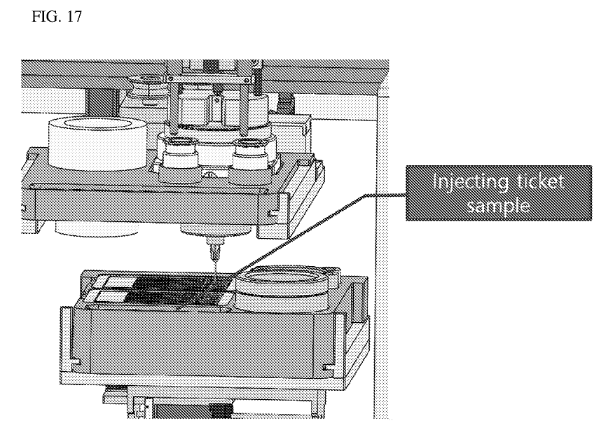

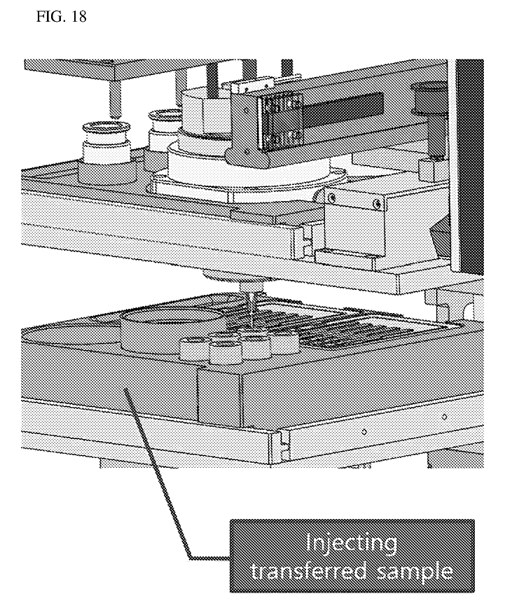

Meanwhile, FIGS. 8 to 18 are views illustrating the operation procedure of the automation device 1000 for identifying and collecting biological agents according to an exemplary embodiment of the present disclosure.

Referring to FIGS. 8 to 18, the automation device 1000 for identifying and collecting biological agents according to an exemplary embodiment of the present disclosure may automatically perform the following steps:

(1) Mounting the filter tray: A filter tray 110 with a dummy filter 160 and a filter assembly 150 mounted thereon is manually mounted to the collection unit 100. Starting from this, the following operations may be automatically performed based on a program or user input.

(2) Alignment for particle collection The filter tray 110 is moved such that the filter assembly 150 is aligned with the collection port 130, and the filter assembly 150 is connected to the collection port 130 to prepare for biological particle collection.

(3) Moving the filter tray: The filter assembly 150 with collected biological particles is separated from the collection port 130, and the filter tray 110 is moved to the identification unit 200.

(4) Connecting dummy filter: The filter tray 110 is moved so that the dummy filter 160 is aligned with the collection port 130, and the dummy filter 160 is connected to the collection port 130.

(5) Mounting the ticket tray: A ticket tray 210, on which the collection container 220, needle assembly 240, and transport sample container 260 are mounted, is manually mounted to the identification unit 200. The manual mounting of the ticket tray 210 may actually be performed before this step, and the operations following the mounting of the ticket tray 210 to the identification unit 200 may be automatically performed based on a program or user input.

(6) Filter alignment: The filter tray 110 is moved such that the filter assembly 150 is aligned and connected to the collection container 220. At this time, a filter housing 155 installed in the filter assembly 150 may rotate 180° by the rotation of the rotation drive unit 125.

(7) Coupling for solution elution: After the filter 156 secured in the filter assembly 150 is automatically rotated, a washing liquid is dropped (dispensed) into the filter assembly 150 to wash biological particles that have moved to the lower part of the filter 156 into the collection container 220.

(8) Moving ticket tray: After the solution containing the washing liquid and biological particles is stored in the collection container 220, the connection between the filter assembly 150 and the collection container 220 is released, and the ticket tray 210 is moved so that the filter assembly 150 is aligned with the needle assembly 240.

(9) Needle assembly connection: After the filter assembly 150 and the needle assembly 240 are aligned, they are connected to each other. Then, the ticket tray 210 is moved again, and the filter assembly 150 is re-aligned with the collection container 220 to draw the solution containing the washing liquid and biological particles from the collection container 220 through the needle assembly 240.

(10) Injecting sample into identification ticket: After the filter assembly 150 automatically draws in the solution containing the washing liquid and biological particles from the collection container 220 via the needle assembly 240, the ticket tray 210 is moved so that the filter assembly 150 is aligned with the identification ticket 230, and the filter assembly 150 injects the drawn solution into the identification ticket 230 via the needle assembly 240.

(11) Injecting sample into transferred sample container: The ticket tray 210 is automatically moved again so that the filter assembly 150 is aligned with the transferred sample container 260, and the filter assembly 150 injects the remaining solution into the transferred sample container 260 via the needle assembly 240.

(12) Image capture and analysis of the sample in the identification ticket: The ticket tray 210 is moved so that the identification ticket 230 is positioned under the camera assembly 320, and the camera assembly 320 captures an image of the identification ticket 230. The image processing module 310 analyzes the image captured by the camera assembly 320 to identify the type and quantity of biological particles.

As described above, the biological agent collection and identification automation apparatus 1000 according to an exemplary embodiment of the present disclosure is capable of continuous operation through an automated process, thereby maximizing high-speed processing and operational efficiency.

In addition, biological particles can be accurately identified through antigen-antibody reactions on the identification ticket 230, and the type and quantity of biological particles can be rapidly and precisely determined through image analysis performed by the camera and the image processing module 310.

Moreover, the use of the dummy filter 160 and the automated coupling/decoupling process prevents contamination and enables hygienic handling of biological samples, thereby maintaining safety and hygiene in the working environment.

Furthermore, the automated operation and detachability of each assembly contribute to the reduction of consumable costs. In particular, the use of disposable needles reduces maintenance costs and enhances the economic efficiency of the apparatus.

In the above, although several preferred embodiments of the present disclosure have been described with some examples, the descriptions of various exemplary embodiments described in the "Specific Content for Carrying Out the Invention" item are merely exemplary, and it will be appreciated by those skilled in the art that the present disclosure can be variously modified and carried out or equivalent executions to the present disclosure can be performed from the above description.

In addition, since the present disclosure can be implemented in various other forms, the present disclosure is not limited by the above description, and the above description is for the purpose of completing the disclosure of the present disclosure, and the above description is just provided to completely inform those skilled in the art of the scope of the present disclosure, and it should be known that the present disclosure is only defined by each of the claims.

List of Reference numbers

100: collection unit

110: filter tray

120: horizontal driving unit

125: rotational driving unit

130: collection port

140: filter holder

150: filter assembly

151: upper case

152: lower case

155: filter housing

155a: rotating shaft

156: filter

157: filter fixing bracket

158: sealing ring

160: dummy filter

170: cleaning solution container

180: vertical driving unit

190: discharge port

195: rotary valve

200: identification unit

210: ticket tray

220: collection container

230: identification ticket

240: needle assembly

241: connector

242: needle

250: cleaning solution inlet

260: transferred sample container

280: ticket tray path

310: image processing module

320: camera assembly

1000: automation device for identifying and collecting biological agents

What is claimed is:

1. An automation device for identifying and collecting biological agents, comprising:
a collection unit including a movable filter tray; and
an identification unit disposed apart from the collection unit and configured to wash and store collected biological agents,
wherein a filter assembly is mounted on the filter tray, the filter assembly being configured to collect biological agents by passing external air through a filter, and
wherein the filter coupled inside the filter assembly is configured to automatically rotate as the filter tray moves to the identification unit for identification of the collected biological agents.

2. The device of claim 1,
wherein the filter tray is configured to move along two axes, including a left-right direction and an up-down direction.

3. The device of claim 1,
wherein the identification unit includes a ticket tray on which a collection container is mounted, and
wherein the ticket tray is configured to move along three axes, including a left-right direction, a front-rear direction, and an up-down direction.

4. The device of claim 1,
wherein the filter assembly comprises:
a filter housing configured to hold the filter; and
a filter fixing bracket to which the filter housing is rotatably coupled,
wherein the filter housing is configured to rotate via a rotating shaft connected to a driving shaft.

5. The device of claim 1,
wherein a collection port is detachably connected to an upper side of the filter assembly, the collection port being configured to collect external air,
wherein a dummy filter is mounted on the filter tray, the dummy filter being spaced apart from the filter assembly, and
wherein, after the filter tray moves to the identification unit, the dummy filter is coupled to the collection port.

6. The device of claim 1,
wherein the identification unit includes a movable ticket tray and a collection container mounted on the ticket tray, and
wherein, when the filter tray moves to the identification unit, the filter assembly and the collection container are brought into alignment and coupled to each other.

7. The device of claim 6,
wherein, after the filter coupled inside the filter assembly automatically rotates, a cleaning solution is dispensed into the filter assembly to wash the biological agents into the collection container.

8. The device of claim 1,
wherein the identification unit includes a movable ticket tray, a collection container mounted on the ticket tray, and a needle assembly, and
wherein the ticket tray is configured to move such that the filter assembly and the needle assembly are brought into alignment and coupled to each other.

9. The device of claim 8,
wherein, after the filter assembly and the needle assembly are coupled to each other, the ticket tray is configured to move such that the filter assembly is brought into alignment with the collection container, and
wherein the needle assembly is configured to aspirate a solution containing biological agents from the collection container.

10. The device of claim 8,
wherein the needle assembly comprises:
a connector configured to be coupled to the filter assembly; and
a needle configured to aspirate and inject a solution,
wherein the needle is detachably attached to the connector.

11. The device of claim 8,
wherein the identification unit includes a movable ticket tray, and
wherein an identification ticket is mounted on the ticket tray, the identification ticket being configured to perform an antigen-antibody reaction for the biological agents.

12. The device of claim 11, further comprising:
a camera assembly configured to capture an image of the identification ticket; and
an image processing module configured to analyze the captured image.

13. The device of claim 12,
wherein the ticket tray is configured to move such that the camera assembly is aligned to capture an image of the identification ticket, and
wherein the image processing module is configured to analyze the image captured by the camera assembly to identify a type and quantity of the biological agents.

14. The device of claim 1,
wherein the identification unit includes a movable ticket tray, and
wherein a transferred sample container is mounted on the ticket tray, the transferred sample container being configured to store and transfer a sample of the biological agents.

15. The device of claim 14,
wherein the ticket tray is configured to move such that the filter assembly is aligned with the transferred sample container, and
wherein the filter assembly is configured to inject the biological agents into the transferred sample container.

16. An automation device for identifying and collecting biological agents, comprising:
a collection port configured to collect external air;
a filter assembly configured to collect biological agents by passing the external air collected by the collection port through a filter;
a filter tray on which the filter assembly is mounted, the filter tray being configured to be movable; and
a ticket tray disposed apart from the filter tray, the ticket tray being movable and having a collection container mounted thereon, the collection container being configured to store the biological agents,
wherein the filter coupled inside the filter assembly is configured to automatically rotate as the filter tray moves to the ticket tray for identification of the collected biological agents.

17. An automation device for identifying and collecting biological agents, comprising:
a collection port configured to collect external air;
a filter assembly detachably connected to the collection port and configured to collect biological agents by passing the external air collected by the collection port through a filter;
a filter tray on which the filter assembly and a dummy filter are mounted, the filter tray being configured to be movable; and
a ticket tray disposed apart from the filter tray, the ticket tray being movable and having a collection container mounted thereon, the collection container being configured to store the biological agents,
wherein, as the filter tray moves to the ticket tray for identification of the biological agents, the filter assembly is disconnected from the collection port, and
wherein, after the filter tray moves to the ticket tray, the dummy filter is connected to the collection port.
